(19) United States
(12) Patent Application Publication
(10) Pub. No.: US 2025/0295787 A1
(43) Pub. Date: Sep. 25, 2025

(54) PRESERVED ETHERIFIED CYCLODEXTRIN DERIVATIVES CONTAINING LIQUID AQUEOUS PHARMACEUTICAL COMPOSITION

(71) Applicant: Boehringer Ingelheim Vetmedica GmbH, Ingelheim am Rhein (DE)

(72) Inventors: Michael AVEN, Mainz (DE); Tim LUKAS, Heidesheim (DE)

(21) Appl. No.: 19/233,379

(22) Filed: Jun. 10, 2025

Related U.S. Application Data
(62) Division of application No. 17/511,220, filed on Oct. 26, 2021, now Pat. No. 12,357,696.
Application No. 17/511,220 is a continuation of application No. 16/047,313, filed on Jul. 27, 2018, now Pat. No. 11,185,590.
Application No. 16/047,313 is a continuation of application No. 14/335,080, filed on Jul. 18, 2014, now Pat. No. 10,071,162.

(30) Foreign Application Priority Data
Jul. 19, 2013 (EP) 13177268.3

(51) Int. Cl. A61K 47/40; A61K 9/00; A61K 9/08; A61K 31/501; A61K 47/69

(52) U.S. Cl. CPC A61K 47/40; A61K 9/0095; A61K 9/08; A61K 31/501; A61K 47/6951

(57) ABSTRACT

A preserved liquid aqueous pharmaceutical composition includes one or more etherified cyclodextrin derivatives, at least one water-soluble preservative, and at least one pharmaceutically active compound which is poorly water-soluble, very poorly water-soluble or water-insoluble. The liquid aqueous pharmaceutical composition provides an acceptable solubility of the pharmaceutically active compound, such as pimobendan, in aqueous solution whereby the water-soluble preservatives retain their effectiveness in the presence of the etherified cyclodextrin derivatives allowing the use in an oral administration form.

### CROSS-REFERENCE TO RELATED APPLICATIONS

This application is a continuation of U.S. patent application Ser. No. 16/047,313, filed Jul. 27, 2018, which is a continuation of U.S. patent application Ser. No. 14/335,080, filed Jul. 18, 2014 (issued as U.S. Pat. No. 10,071,162), which claims priority to EP Patent Application No. 13 177 268.3, filed Jul. 19, 2013, the disclosures of which are incorporated herein by reference in their entireties.

### FIELD OF THE INVENTION

The invention relates to the field of medicine, particularly veterinary medicine. In particular, the invention relates to a novel preserved liquid aqueous pharmaceutical composition comprising one or more etherified cyclodextrin derivatives, one or more water-soluble preservatives and at least one pharmaceutically active compound.

### BACKGROUND OF THE INVENTION

Cyclodextrins are cyclic oligosaccharides containing six, seven, or eight (α-1,4)-linked D-glucopyranoside units resulting in alpha (α)-, beta (β)- and gamma (γ)-cyclodextrins. In general, cyclodextrins are pharmaceutical excipients that can solubilize various poorly soluble drugs/molecules through the formation of water-soluble drug-cyclodextrin complexes (Loftsson T et al., Journal of Pharmaceutical Sciences 2012, 101 (9): 3019-3032). More specifically, cyclodextrins in aqueous solution form inclusion complexes with water-insoluble or poorly soluble drugs by taking up the lipophilic moiety of the drug molecule into the cavity of the cyclodextrin, which is hydrophobic (Brewster M E et al., Advanced Drug Delivery Reviews 2007, 59:645-666). However, non-inclusion drug-cyclodextrin complexes can also be formed. The higher the cyclodextrin concentration increases, the higher the formation of aggregates of cyclodextrin molecules and self-assembled complexes. A further aspect with cyclodextrin containing pharmaceutical compositions is the formation of self-assembled complexes and/or formation of aggregates (Messner M et al., International Journal of Pharmaceutics 2011, 408:235-247). Excipients that solubilize and stabilize such aggregates include small ionized molecules such as salts of organic acids and bases.

A substantial problem with pharmaceutical compositions including cyclodextrins is to produce pharmaceutical compositions which are preserved against microbial growth. Such preserved compositions are particularly important for storage of containers containing multiple-dose compositions. Typical preservatives are relatively ineffective at normal concentrations in such compositions, as compositions including such preservatives are unable to meet or pass standard preservative efficacy tests (for example USP <51> or Pharm. Eur. 5.1.3. It is believed that the preservative forms a complex with cyclodextrin and consequently is rendered ineffective or has reduced effectiveness as a preservative. Thus, the preservative loses its full activity by complex formation. The formation of these complexes between preservative and cyclodextrin further reduce the solubility of the active drug substance (Loftsson T et al., Drug Development and Industrial Pharmacy 1992, 18 (13): 1477-1484).

Certain etherified β-cyclodextrin derivatives are known to improve solubility of sparingly soluble drugs, see WO 85/02767. However, in WO 85/02767 only the use of etherified β-cyclodextrin derivatives up to a concentration of 10% is described. A molar ratio of drug to etherified β-cyclodextrin derivative of 1:6 to 4:1 was contemplated. The solubility of flubendazol within the above given ratio was only increased by a factor 30. However, those formulations are not suitable for the preparation of pharmaceutical compositions comprising substituted benzimidazole derivatives, such as pimobendan.

Further prior art is as follows:
US 2004/152664 is directed to compositions comprising cyclodextrin derivatives and prednisolone.
WO 2004/089418 deals with a fluoroquinolone comprising aqueous formulations of a pH between 4 and 7.
EP 1 920 785 discloses a liquid preparation comprising a complex of pimobendan and cyclodextrin.
Brewster M E at al. (Advanced Drug Delivery Reviews 2007, 59(7): 645-666) describe cyclodextrins as pharmaceutical solubilizers.
Bassani V L et al. (Journal of Inclusion Phenomena and Molecular Recognition in Chemistry, 1996, 25(1-3): 149-152) refer to the enhanced water-solubility of albendazole by hydroxypropyl-β-cyclodextrin complexation.

The article of Piel G and co-workers (Sciences Techniques et Pratiques STP Pharma Pratiques 1999, 9(3): 257-260) is directed to the development of a parenteral and an oral formulation of albendazole with cyclodextrins.

This enables the development of a pharmaceutical composition for parenteral use but due to the reduced shelf-life of unpreserved compositions, it does not enable the development of a pharmaceutical multiple-dose composition for oral use. Due to the risk of severe tolerance problems and also due to concerns by pet-owners that inflammation in the subcutis following injections is considered to be a risk factor in the development of sarcomas, it is highly desirable to develop an oral pharmaceutical composition.

Due to some animals' intense sense of taste, it is particularly difficult to formulate a medication that can be administered orally and which the animal accepts resulting in an easy to use medication for animals, in particular companion animals, such as dogs, cats and horses (sufficiently good palatability).

The objective underlying the present invention is therefore to provide a pharmaceutical composition which overcomes the problems of the prior art as described above. Particularly, a pharmaceutical composition containing a sparingly water-soluble pharmaceutical active compound at palatable pH values (e.g. ≥pH 3) shall be provided to be administered in adequate form to a subject in need thereof.

### SUMMARY OF THE INVENTION

It is therefore provided a preserved liquid aqueous pharmaceutical composition comprising one or more etherified cyclodextrin derivatives; one or more water-soluble preservatives; preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; parabenes and salts thereof, preferably methylparabene, ethylparabene, propylparabene, butylparabene, butylparabene sodium; or combinations thereof; more preferably selected from the group of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof; and at least one pharmaceutically active compound which is poorly water-soluble, very poorly water-soluble or water-insoluble; wherein preferably the solubility of the at least one pharmaceutically active compound in water in the range of 15 to 25° C. is defined as follows: the at least one pharmaceutically active compound is poorly water-soluble if more than 100 ml of water per gram compound have to be used; it is very poorly water-soluble if more than 1000 ml of water per gram compound have to be used; and it is water-insoluble if more than 10,000 mL water per gram compound have to be used to solubilize the compound; and preferably with the proviso that corticosteroids, in particular prednisolone and its prodrug prednisolone acetate (see US 2004/152664), and fluoroquinolones, in particular ciprofloxacin, gatifloxacin, moxifloxacin, sitafloxacin, lomefloxacin, grepafloxacin, gemifloxacin, norfloxacin, ofloxacin, levofloxacin, trovafloxacin and the like (see WO 2004/089418), are independently from each other excluded as pharmaceutically active compound which is poorly water-soluble, very poorly water-soluble or water-insoluble.

The present invention is also directed to the liquid pharmaceutical composition for use in a method for treating a subject in need of such treatment, preferably an animal, in particular a companion animal, even more preferred horse, dog or cat, guinea pig, hamster, cattle, goat, sheep, in particular cat or dog, selected from among the indications: heart diseases, particularly a hypertrophic cardiomyopathy, more particularly heart failure (HF), congestive heart failure (CHF), acute CHF, decompensated endocardiosis (DCE), dilated cardiomyopathy (DCM), asymptomatic (occult) CHF, asymptomatic DCM, hypertrophic cardiomyopathy (HCM), restricted cardiomyopathy (RCM), and heart failure due to HCM, RCM, DCM and/or UCM.

It is also disclosed a process for producing the pharmaceutical composition comprising the steps, adding at least one pharmaceutically active compound, one or more etherified cyclodextrin derivatives, one or more water-soluble preservatives, optionally one or more antioxidants and optionally at least one water-soluble polymer to water and mixing under stirring, adjusting the pH value using a pH adjustment agent, wherein preferably the one or more water-soluble preservatives are added after the addition of the at least one pharmaceutically active compound.

Subject of the present invention is also a kit of parts that comprises:
a. a preserved liquid aqueous pharmaceutical composition according to the present invention; and
b. a package leaflet including the information that the pharmaceutical composition is to be used for the prevention and/or treatment of a heart disease, preferably heart failure and/or hypertrophic cardiomyopathy, in a subject in need of such prevention or treatment.

It is unexpected that the pharmaceutical composition of the present invention can overcome the deficiencies of prior art. The liquid aqueous pharmaceutical compositions for oral administration comprising sparingly or not water-soluble pharmaceutically active compounds, such as pimobendan, known from prior art are usually not suitable due to the low concentration of pharmaceutically active compound normally achieved.

A known pharmaceutically active compound is pimobendan (4,5-dihydro-6-[2-(4-methoxyphenyl)-1H-benzimidazol-5-yl]-5-methyl-3 (2H)-pyridazinone) disclosed in EP 0 008 391, herein incorporated by reference in its entirety, and having the formula:

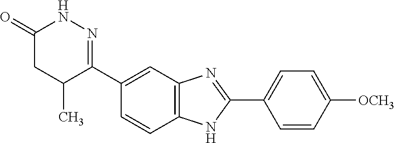 [SMILES: COc1ccc(-c2nc3cc(C4=NNC(=O)CC4C)ccc3[nH]2)cc1]

Pimobendan is a well-known compound for the treatment of congestive heart failure (CHF) originating for example from dilated cardiomyopathy (DCM) or decompensated endocardiosis (DCE) in animals, especially dogs (WO 2005/092343). Furthermore, pimobendan is also used for the treatment of hypertrophic cardiomyopathy in cats (WO 2010/060874). Pimobendan is also approved as a drug product for cardiovascular treatment of humans.

As already described in EP 0 439 030 and WO 2005/08467, pimobendan drug substance is insoluble in water: 1 g drug substance is soluble in more than 10,000 mL. At pH 7 the solubility of pimobendan is only about 0.1 mg per 100 mL.

The solubility of pimobendan in aqueous solutions is depends on the pH. The solubility of pimobendan is significantly higher at pH 1 to 2.5 than at higher pH values (pH ≥3.0). However, the local tolerance and palatability as well as the chemical stability of such a formulation are not acceptable. This is due to the fact that the target dose would require a drug concentration in solution which can only be achieved by a pH of about pH 2.5 and lower. However, the concentration has to be significantly higher, resulting in a low volume that the animal will have to swallow, than is possible at pH ≥3.0 in simple aqueous solutions. Accordingly, a pimobendan formulation comprising up to 1.5 mg/mL of pimobendan would need an increase in solubility at pH 7 by a factor of about 1000 to 1500, not achieved in prior art formulations for oral administration up to now.

On the contrary, the preserved liquid aqueous pharmaceutical compositions according to the present invention comprising at least one pharmaceutically active compound which is poorly water-soluble, very poorly water-soluble or water-insoluble with the assistance of one or more etherified cyclodextrin derivatives provides an acceptable solubility of the pharmaceutically active compound such as pimobendan in aqueous solution. Thereby, an acceptable concentration of the pharmaceutically active compound is present allowing for use in an oral administration form.

Further, the one or more water-soluble preservatives present assure an acceptable efficacy of microbial preservation over the required shelf life of the pharmaceutical composition of the present invention.

Furthermore, and unexpectedly, the above water-soluble preservatives retain their effectiveness in the presence of the etherified cyclodextrin derivative(s), i.e. the included water-soluble preservatives do have a substantial preserving efficacy in the presence of cyclodextrin components.

Since the preserved liquid aqueous pharmaceutical compositions according to the present invention may be formulated for oral administration the disadvantageous side effects of parenteral administration such as inflammation in the subcutis following injections may be avoided. In addition, the composition does not have to be given by a veterinarian, as is the case for parenteral administration.

Also the palatability if administered to animal patients is found to be good apparently due to a high concentration of well-palatable etherified cyclodextrin-derivatives present in the pharmaceutical composition of the present invention.

Moreover, the addition of some excipients such as water-soluble polymers and/or antioxidants have been found to be advantageous in order to further increase the concentration of the pharmaceutically active compound to be used and/or to further stabilize the liquid pharmaceutical composition without interfering with the preservative effectiveness of the water-soluble preservatives.

### DETAILED DESCRIPTION OF THE INVENTION

Before the embodiments of the present invention are described in further details it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The present invention is based on the surprising unexpected observation that a pharmaceutical composition comprising one or more etherified cyclodextrin derivatives and at least one pharmaceutically active compound can be preserved, without occurrence of the above described deficiencies, in particular that included water-soluble preservatives do have a substantial preserving efficacy in the presence of cyclodextrin components.

According to the present invention a preserved liquid aqueous pharmaceutical composition is provided. The term “aqueous” is to be understood in the meaning that the pharmaceutical composition contains water as a solvent, whereby also one or more additional solvents may be optionally present. According to one preferred embodiments water is the only solvent of such pharmaceutically composition.

The liquid aqueous pharmaceutical composition comprises at least one pharmaceutically active compound which is poorly water-soluble, very poorly water-soluble or water-insoluble. According to the European Pharmacopoeia the solubility of a compound in water in the range of 15 to 25° C. is defined as follows:

| | Solvent in mL per gram compound |
|---|---|
| Very readily soluble | <1 |
| Readily soluble | from 1 to 10 |
| Soluble | from >10 to 30 |
| Hardly soluble | from >30 to 100 |
| Poorly soluble | from >100 to 1,000 |
| Very poorly soluble | from >1,000 to 10,000 |
| Water-insoluble | >10,000. |

Thus, according to the present invention the at least one pharmaceutically active compound is poorly water-soluble, very poorly water-soluble or water-insoluble. Preferably the at least one pharmaceutically active compound is poorly water-soluble if more than 100 mL of water per gram compound have to be used; it is very poorly water-soluble if more than 1,000 mL of water per gram compound must be used; and it is water-insoluble if more than 10,000 mL water per gram compound have to be used to solubilize the compound.

The at least one pharmaceutically active compound is preferably a benzimidazole derivative. The benzimidazole derivative is preferably a substituted benzimidazole. The term “substituted benzimidazole” as used herein means, but is not limited to thiabendazol, fuberidazol, oxibendazol, parbendazol, cambendazol, mebendazol, fenbendazol, flubendazol, albendazol, oxfendazol, nocodazol, astemisol and pimobendan, pharmaceutically acceptable salts, derivatives, metabolites or prodrugs thereof. Most preferably, the term benzimidazole derivative as used herein means pimobendan, or any pharmaceutically acceptable salts thereof.

In another aspect the at least one pharmaceutically active compound is preferably an oxicam derivative. The oxicam derivative is preferably a substituted oxicam. The term “substituted oxicam” as used herein means, but is not limited to ampiroxicam, droxicam, lornoxicam, piroxicam, tenoxicam and meloxicam, pharmaceutically acceptable salts, derivatives, metabolites or prodrugs thereof. Most preferably, the term oxicam derivative as used herein means meloxicam, or any pharmaceutically acceptable salts thereof.

In another aspect the at least one pharmaceutically active compound is preferably an imidazolinone derivative. The imidazolinone derivative is preferably a substituted imidazolinone. The term “substituted imidazolinone” as used herein means, but is not limited to 1-(4-chlorophenyl)-4-(4-morpholinyl)-2,5-dihydro-1H-imidazol-2-one (imepitoin), pharmaceutically acceptable salts, derivatives, metabolites or prodrugs thereof. Most preferably, the term imidazolinone derivative as used herein means 1-(4-chlorophenyl)-4-(4-morpholinyl)-2,5-dihydro-1H-imidazol-2-one (imepitoin), or any pharmaceutically acceptable salts thereof.

In another aspect the at least one pharmaceutically active compound is preferably a glucopyranosyl-substituted benzene derivative. The glucopyranosyl-substituted benzene derivative is preferably a substituted glucopyranosyl-substituted benzene derivative. The term “substituted glucopyranosyl-substituted benzene derivative” as used herein means, but is not limited to 1-cyano-2-(4-cyclopropyl-benzyl)-4-(β-D-glucopyranos-1-yl)-benzene, pharmaceutically acceptable salts, derivatives, metabolites or prodrugs thereof. Most preferably, the term glucopyranosyl-substituted benzene derivative as used herein means 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline.

The liquid aqueous pharmaceutical composition according to the present invention contains the at least one pharmaceutically active compound as disclosed herein, particularly in form of a substituted benzimidazole, more particularly pimobendan, preferably in the range of from 0.01 g/100 mL to 1 g/100 mL, more preferably from 0.05 g/100 mL to 0.5 g/100 mL, most preferably from 0.1 g/100 mL to 0.25 g/100 mL.

Due to the low aqueous solubility of the pharmaceutically active compound as disclosed herein, preferably a substituted benzimidazole, such as pimobendan, at pH values that are acceptable for an oral pharmaceutical composition, one or more solubilizing excipients need to be added to the formulation.

In the present invention such solubilizing excipients are one or more etherified cyclodextrin derivatives.

The liquid aqueous pharmaceutical composition according to the present invention contains the one or more etherified cyclodextrin derivatives preferably in the range of from 5 g/100 mL to 40 g/100 mL more preferably from 10 g/100 mL to 35 g/100 mL, most preferably from 20 g/100 mL to 35 g/100 mL per one etherified cyclodextrin derivative.

The term “etherified cyclodextrin derivative” as used herein includes but is not limited to alpha-, beta- or gamma-cyclodextrin ethers. Preferably the one or more etherified cyclodextrin derivatives as used herein means etherified β-cyclodextrins, more preferably of the chemical formula I:

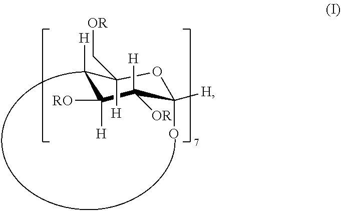

in which the residues R are independently from each other hydroxyalkyl groups and part of the residues R may optionally independently from each other be alkyl groups. A partially etherified β-cyclodextrin of formula I is preferably used, in which the residues R are independently from each other hydroxyethyl, hydroxypropyl or dihydroxypropyl groups. Optionally, part of the residues R may for instance be methyl or ethyl groups.

The use of partially methylated β-cyclodextrins with 7 to 14 methyl groups in the β-cyclodextrin molecule as they are known from DE 31 18 218 does not fall under the present invention.

Partial ethers of β-cyclodextrin comprising only alkyl groups, such as methyl, ethyl and the like, may be particularly suitable in accordance with the invention if they have a low degree of substitution, preferably as defined below of 0.05 to 0.2.

Even more preferably, the one or more etherified cyclodextrin derivatives as used herein are hydroxyethyl-β-cyclodextrin, hydroxypropyl-β-cyclodextrin, dihydroxypropyl-β-cyclodextrin, sulfobutyl-ether-β-cyclodextrin.

Most preferably, the one or more etherified cyclodextrin derivatives as used herein are hydroxypropyl-β-cyclodextrin (HPBCD), referred to as hydroxypropylbetadex in the European Pharmacopoeia. Hydroxypropyl-β-cyclodextrin (HPBCD) of pharmaceutical grade is marketed for example under the trademark CAVASOL® W7 HP Pharma and can be ordered from Wacker, Germany.

Beta-cyclodextrin is a compound with ring structure consisting of 7 anhydro glucose units; it is also referred to as cycloheptaamylose. Each of the 7 glucose rings contains in 2-, 3-, and 6-position three hydroxy groups which may be etherified. In the partially etherified one or more β-cyclodextrin derivatives used according to the invention only part of these hydroxy groups is etherified with hydroxyalkyl groups and optionally further with alkyl groups. When etherifying with hydroxyalkyl groups, which can be carried out by reaction with the corresponding alkylene oxides, the degree of substitution is stated as molar substitution (MS), viz. in mole alkylene oxide per anhydroglucose unit (compare U.S. Pat. No. 3,459,731, column 4). In the hydroxyalkyl ethers of β-cyclodextrin used in accordance with the invention the molar substitution is preferably between 0.05 and 10, more preferably between 0.2 and 2. Particularly preferred is a molar substitution of about 0.40 to about 1.50. The etherification with alkyl groups may be stated directly as degree of substitution (DS) per glucose unit which as stated above is 3 for complete substitution. Partially etherified β-cyclodextrins are used within the invention which preferably comprise besides hydroxyalkyl groups also alkyl groups, especially methyl or ethyl groups, up to a degree of substitution of 0.05 to 2.0, more preferably 0.2 to 1.5. Most preferably the degree of substitution with alkyl groups is between about 0.5 and about 1.2.

As solubilizing excipient hydroxypropyl-β-cyclodextrin (HPβCD) showed very advantageous effects and resulted in the largest increase in solubility of a pharmaceutically active compound to be used such as pimobendan or a pharmaceutically acceptable salt thereof.

To prevent microbial growth in the solution during the in-use period one or more water-soluble preservatives are added to the liquid aqueous pharmaceutical composition. Therefore, the liquid aqueous pharmaceutical composition of the present invention comprises one or more water-soluble preservatives. The one or more water-soluble preservatives are preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; parabenes and salts thereof, preferably methylparabene, ethylparabene, propylparabene, butylparabene, butylparabene sodium; or combinations thereof. In a more preferred embodiment, the one or more water-soluble preservatives are selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof. Particularly preferred is sorbic acid or salts thereof.

The liquid aqueous pharmaceutical composition according to the present invention contains the one or more water-soluble preservatives preferably in the range of from 0.05 g/100 mL to 3.0 g/100 mL, more preferably from 0.10 g/100 mL to 1.0 g/100 mL, most preferably from 0.20 g/100 mL to 0.40 g/100 mL.

The above disclosed water-soluble preservatives do not displace the pharmaceutically active compound from the cyclodextrin complex. Furthermore, and unexpectedly, the above water-soluble preservatives retain their effectiveness in the presence of the etherified cyclodextrin derivative.

Therefore, the water-soluble preservatives as listed above allow the provision of a preserved cyclodextrin-containing pharmaceutical composition which is particularly suitable for oral and/or parenteral use in veterinary medicine, preferably oral use.

Thus, according to one aspect, the present invention relates to a preserved liquid aqueous pharmaceutical composition comprising one or more etherified cyclodextrin derivatives, one or more water-soluble preservatives and at least one pharmaceutically active compound as disclosed herein, particularly in form of a substituted benzimidazole, more particularly pimobendan, wherein the one or more etherified cyclodextrin derivative is selected from the group consisting of: alpha-, beta-, and/or gamma-cyclodextrin ether.

According to a further aspect, the present invention relates to a preserved liquid aqueous pharmaceutical composition as described above, comprising one or more etherified cyclodextrin derivatives, one or more water-soluble preservatives and at least one pharmaceutically active compound as disclosed herein, particularly in form of a substituted benzimidazole, more particularly pimobendan, wherein the one or more etherified cyclodextrin derivative is etherified β-cyclodextrin. Preferably, that etherified β-cyclodextrin is hydroxyethyl-β-cyclodextrin, hydroxypropyl-β-cyclodextrin, or dihydroxypropyl-β-cyclodextrin. Even more preferably, that etherified β-cyclodextrin is hydroxypropyl-β-cyclodextrin (HPBCD), referred to as hydroxypropylbetadex in the European Pharmacopoeia.

The preserved liquid aqueous pharmaceutical composition according to the present invention may contain one or more excipients. The one or more excipients can be selected from the group consisting of an antioxidant, a water-soluble polymer, buffer, pH adjustment agent, colorants or taste-masking ingredients including flavors.

Preferably at least one water-soluble antioxidant and/or at least one water-soluble polymer may be used. More preferably, at least one water-soluble antioxidant and at least one water-soluble polymer are added as excipients.

In a preferred embodiment, the liquid aqueous pharmaceutical composition of the present invention further comprises at least one water-soluble antioxidant and/or at least one water-soluble polymer, more preferably at least one water-soluble antioxidant and at least one water-soluble polymer.

Thus, according to a preferred embodiment the present invention is directed to a preserved liquid aqueous pharmaceutical composition comprising one or more etherified cyclodextrin derivatives, one or more water-soluble preservatives; preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; parabenes and salts thereof, preferably methylparabene, ethylparabene, propylparabene, butylparabene, butylparabene sodium; or combinations thereof, more preferably selected from the group of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof; at least one pharmaceutically active compound which is poorly water-soluble, very poorly water-soluble or water-insoluble; preferably with the proviso that corticosteroids, in particular prednisolone and its prodrug prednisolone acetate (see US 2004/152664), and fluoroquinolones, in particular ciprofloxacin, gatifloxacin, moxifloxacin, sitafloxacin, lomefloxacin, grepafloxacin, gemifloxacin, norfloxacin, ofloxacin, levofloxacin, trovafloxacin and the like (see WO 2004/089418), are independently from each other excluded as pharmaceutically active compound which is poorly water-soluble, very poorly water-soluble or water-insoluble; and at least one water-soluble antioxidant.

According to the invention it is preferred that the liquid aqueous pharmaceutical composition comprises at least one water-soluble antioxidant because a combination of a water-soluble preservative and an antioxidant in order to stabilize the water-soluble preservative is particularly preferred. Only a small number of antioxidants are known which are water-soluble and come into question, such as free-radical scavengers, reduction agents and/or chelating agents. Water-soluble antioxidants that can be used comprise ascorbic acid or pharmaceutically acceptable salts thereof, particularly sodium ascorbate; citric acid (anhydrous and/or monohydrate) or pharmaceutically acceptable salts thereof, particularly sodium citrate; erythorbic acid; fumaric acid; malic acid; monothioglycerol; phosphoric acid; sodium metabisulfite; potassium metabisulfite; propionic acid; sodium bisulfite; sodium sulfite; resveratrol, butylhydroxyanisol, gallate derivatives, particularly propylgallate, or combinations thereof, preferably ascorbic acid or pharmaceutically acceptable salts thereof, citric acid (anhydrous and/or monohydrate) or pharmaceutically acceptable salts thereof, sodium metabisulfite, or potassium metabisulfite. Particularly preferred is ascorbic acid or pharmaceutically acceptable salts thereof.

A preservative system comprising one or more water-soluble preservatives preferably in form of an acid or salt thereof and at least one water-soluble antioxidant has been shown to be particularly efficient in preserving the above described liquid aqueous pharmaceutical compositions without having a negative effect on the concentration of the pharmaceutically active compound in the pharmaceutical compositions. Accordingly, in a preferred embodiment, the liquid aqueous pharmaceutically composition of the invention comprises one or more water-soluble preservatives and at least one water-soluble antioxidant.

It was found that in particular sorbic acid or a salt thereof shows advantageous characteristics and preserves the liquid aqueous pharmaceutical composition adequately, albeit at a higher concentration than in solutions not containing a cyclodextrin. From the viewpoint of antimicrobial preservation the pH range of 2.5 to 4.5, in particular 3.5, is advantageous of (1) being in the acidic range (improved antimicrobial activity even without a preservative) and (2) being well below the acid dissociation constant (pKₐ) value of 4.75 for sorbic acid. Only at pH values below pKₐ most of the sorbic acid is present in the protonated (uncharged) state, which is necessary for diffusion through the cell membrane of bacteria and fungi.

Furthermore, the presence of at least one water-soluble antioxidant has a positive influence on the pharmaceutical composition of the present invention:

The water-soluble antioxidant, preferably ascorbic acid or salts thereof, was found to chemically stabilize the one or more water-soluble preservatives, for example sorbic acid or salts thereof, in the formulation. Furthermore, the solubility of the one or more water-soluble preservatives could be increased if at least one antioxidant was present. Tests showed an increase in solubility of sorbic acid by about 0.25% (m/V) by the addition of ascorbic acid.

Furthermore, some water-soluble preservatives such as sorbic acid and potassium sorbate are sensitive to oxidation so that at least one antioxidant should preferably be added.

Small amounts of antioxidant may have a benefit for the pharmaceutical composition according to the present invention.

In a further aspect the liquid aqueous pharmaceutical composition according to the present invention comprises at least one water-soluble antioxidant preferably in the range of from 0.2 g/100 mL to 2.0 g/100 mL, in particular from 0.3 g/100 mL to 1.0 g/100 mL.

In a further aspect the liquid aqueous pharmaceutical composition according to the present invention comprises a ratio of water-soluble preservative and antioxidant preferably being from 0.1 to 10, in particular from 0.1 to 1.5, most preferably from 0.2 to 0.8.

According to the invention it has been found that the concentration of the pharmaceutically active compound that is dissolved with the assistance of one or more etherified cyclodextrin derivatives may be further increased by the addition of at least one water-soluble polymer.

It has been found that the water-soluble polymer does not influence the preservative effectiveness. Furthermore, the described formation of self-assembled complexes and/or formation of aggregates may be further reduced or completely prevented by excipients that solubilize and stabilize such aggregates, e.g. water-soluble polymers such as cellulose derivatives.

In addition, inclusion of such water-soluble polymers in the formulation can be used to optimize the viscosity of the oral solution to ease dosing for example from a plastic syringe.

According to the invention the at least one water-soluble polymer has preferably a molar mass of 5,000 to 500,000 g/mol, more preferably 10,000 to 300,000 g/mol, even more preferred 15,000 to 200,000 g/mol, even more preferred 20,000 to 200,000 g/mol. Examples for said water soluble polymer are hydroxypropyl methylcellulose (hypromellose, HPMC), hydroxypropyl cellulose, carboxymethylcellulose, hydroxyethyl cellulose, hydroxyethylmethyl cellulose, ethylcellulose, methylcellulose, polyvinylpyrrolidone, polyvinylacetate as well as combinations or copolymers thereof, preferably hydroxypropyl methylcellulose (hypromellose).

The liquid aqueous pharmaceutical composition according to the present invention optionally contains the at least one water-soluble polymer preferably in the range of from 0.01 g/100 mL to 0.75 g/100 mL, more preferably from 0.02 g/100 mL to 0.50 g/100 mL, most preferably from 0.05 g/100 mL to 0.30 g/100 mL.

Thus, according to a preferred embodiment the present invention is directed to a preserved liquid aqueous pharmaceutical composition comprising one or more etherified cyclodextrin derivatives; one or more water-soluble preservatives; preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; parabenes and salts thereof, preferably methylparabene, ethylparabene, propylparabene, butylparabene, butylparabene sodium; or combinations thereof, more preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof; at least one pharmaceutically active compound which is poorly water-soluble, very poorly water-soluble or water-insoluble; preferably with the proviso that corticosteroids, in particular prednisolone and its prodrug prednisolone acetate (see US 2004/152664), and fluoroquinolones, in particular ciprofloxacin, gatifloxacin, moxifloxacin, sitafloxacin, lomefloxacin, grepafloxacin, gemifloxacin, norfloxacin, ofloxacin, levofloxacin, trovafloxacin and the like (see WO 2004/089418), are independently from each other excluded as pharmaceutically active compound which is poorly water-soluble, very poorly water-soluble or water-insoluble; and at least one water-soluble polymer.

According to the invention the pH of the pharmaceutical composition for oral use has preferably a pH value of 2 to 10, more preferably 3 to 10, more preferably 3 to 8, more preferably 3.1 to 8, more preferably 3 to 7, even more preferably 3.2 to 7, even more preferably 2.5 to 5, most preferably 3 to 5. Particularly preferred is pH 3.3 to 6, particularly 3.4 to 5, especially 3.4 to 4. By using the lowest preferred, but still acceptable pH value, it is possible to further increase the solubility of the pharmaceutically active compound as disclosed herein, such as pimobendan, compared to that at higher pH values. Besides the better solubility of the pharmaceutically active compound compared to higher pH values, the lower pH value range has the further advantage of improved preservative efficacy. An improved preservative efficacy results in a lower concentration of a given preservative which is required to achieve an adequate preservative effect.

According to a further preferred embodiment the present invention is directed to a preserved liquid aqueous pharmaceutical composition comprising one or more etherified cyclodextrin derivatives; one or more water-soluble preservatives; preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; parabenes and salts thereof, preferably methylparabene, ethylparabene, propylparabene, butylparabene, butylparabene sodium; or combinations thereof, more preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof; at least one pharmaceutically active compound which is poorly water-soluble, very poorly water-soluble or water-insoluble; preferably with the proviso that corticosteroids, in particular prednisolone and its prodrug prednisolone acetate (see US 2004/152664), and fluoroquinolones, in particular ciprofloxacin, gatifloxacin, moxifloxacin, sitafloxacin, lomefloxacin, grepafloxacin, gemifloxacin, norfloxacin, ofloxacin, levofloxacin, trovafloxacin and the like (see WO 2004/089418), are independently from each other excluded as pharmaceutically active compound which is poorly water-soluble, very poorly water-soluble or water-insoluble; at least one water-soluble antioxidant; and at least one water-soluble polymer.

According to a further aspect, the present invention relates to a liquid aqueous pharmaceutical composition as described above, comprising at least one pharmaceutically active compound in the form of at least one substituted benzimidazole or a pharmaceutically acceptable salt thereof or a substituted oxicam or a pharmaceutically acceptable salt thereof or a substituted imidazolinone or a pharmaceutically acceptable salt thereof or a substituted glucopyranosyl-substituted benzene derivative or a pharmaceutically acceptable form and/or salt thereof, one or more etherified cyclodextrin derivatives in the form of etherified β-cyclodextrin, one or more water-soluble preservatives, optionally at least one water-soluble polymer and optionally at least one water-soluble antioxidant.

Therefore, the present invention preferably relates to a liquid aqueous pharmaceutical composition as described above, comprising:
a. at least one pharmaceutically active compound in the form of a substituted benzimidazole or a pharmaceutically acceptable salt thereof, preferably thiabendazol, fuberidazol, oxibendazol, parbendazol, cambendazol, mebendazol, fenbendazol, flubendazol, albendazol, oxfendazol, nocodazol, astemisol or pimobendan, or pharmaceutical acceptable salts thereof, more preferably pimobendan or a pharmaceutically acceptable salt thereof;
b. one or more etherified cyclodextrin derivatives in the form of etherified β-cyclodextrin, preferably hydroxyethyl-β-cyclodextrin, hydroxypropyl-β-cyclodextrin, dihydroxypropyl-β-cyclodextrin, more preferably hydroxypropyl-β-cyclodextrin (HPBCD);
c. one or more water-soluble preservatives, preferably selected from the group consisting of sorbic acid or salts thereof, benzoic acid or salts thereof, benzalkonium chloride, benzethonium chloride, cetylpyridinium chloride, sodium metabisulfite, sodium acetate; parabenes and salts thereof, preferably methylparabene, ethylparabene, propylparabene, butylparabene, butylparabene sodium; or combinations thereof, more preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof, most preferably sorbic acid or salts thereof;
d. optionally, but according to a preferred embodiment, at least one water-soluble antioxidant, preferably ascorbic acid or a salt thereof; citric acid (anhydrous and/or monohydrate) or a salt thereof; sodium metabisulfite, potassium metabisulfite or resveratrol; and
e. optionally, but according to a preferred embodiment, at least one water-soluble polymer with a molar mass of 5,000 to 500,000 g/mol, preferably 10,000 to 300,000 g/mol, even more preferred 15,000 to 200,000 g/mol, even more preferred 20,000 to 200,000 g/mol, preferably hydroxypropyl methylcellulose, hydroxypropyl cellulose, or methylcellulose, more preferably hydroxypropyl methylcellulose (hypromellose).

Therefore, the present invention preferably relates to a liquid aqueous pharmaceutical composition as described above, comprising:
a. at least one pharmaceutically active compound in the form of a substituted oxicam or a pharmaceutically acceptable salt thereof, preferably ampiroxicam, droxicam, lornoxicam, piroxicam, tenoxicam and meloxicam, or pharmaceutical acceptable salts thereof, more preferably meloxicam or a pharmaceutically acceptable salt thereof;
b. one or more etherified cyclodextrin derivatives in the form of etherified β-cyclodextrin, preferably hydroxyethyl-β-cyclodextrin, hydroxypropyl-β-cyclodextrin, dihydroxypropyl-β-cyclodextrin, more preferably hydroxypropyl-β-cyclodextrin (HPBCD);
c. one or more water-soluble preservatives, preferably selected from the group consisting of sorbic acid or salts thereof, benzoic acid or salts thereof, benzalkonium chloride, benzethonium chloride, cetylpyridinium chloride, sodium metabisulfite, sodium acetate; parabenes and salts thereof, preferably methylparabene, ethylparabene, propylparabene, butylparabene, butylparabene sodium; or combinations thereof, more preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof, most preferably sorbic acid or salts thereof;
d. optionally, but according to a preferred embodiment, at least one water-soluble antioxidant, preferably ascorbic acid or a salt thereof; citric acid (anhydrous and/or monohydrate) or a salt thereof; sodium metabisulfite, potassium metabisulfite or resveratrol; and
e. optionally, but according to a preferred embodiment, at least one water-soluble polymer with a molar mass of 5,000 to 500,000 g/mol, preferably 10,000 to 300,000 g/mol, even more preferred 15,000 to 200,000 g/mol, even more preferred 20,000 to 200,000 g/mol, preferably hydroxypropyl methylcellulose, hydroxypropyl cellulose, or methylcellulose, more preferably hydroxypropyl methylcellulose (hypromellose).

Therefore, the present invention preferably relates to a liquid aqueous pharmaceutical composition as described above, comprising:
a. at least one pharmaceutically active compound in the form of a substituted imidazolinone or a pharmaceutically acceptable salt thereof, preferably 1-(4-chlorophenyl)-4-(4-morpholinyl)-2,5-dihydro-1H-imidazol-2-one (imepitoin) or a pharmaceutically acceptable salt thereof;
b. one or more etherified cyclodextrin derivatives in the form of etherified β-cyclodextrin, preferably hydroxyethyl-β-cyclodextrin, hydroxypropyl-β-cyclodextrin, dihydroxypropyl-β-cyclodextrin, more preferably hydroxypropyl-β-cyclodextrin (HPBCD);
c. one or more water-soluble preservatives, preferably selected from the group consisting of sorbic acid or salts thereof, benzoic acid or salts thereof, benzalkonium chloride, benzethonium chloride, cetylpyridinium chloride, sodium metabisulfite, sodium acetate; parabenes and salts thereof, preferably methylparabene, ethylparabene, propylparabene, butylparabene, butylparabene sodium; or combinations thereof, more preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof, most preferably sorbic acid or salts thereof;
d. optionally, but according to a preferred embodiment, at least one water-soluble antioxidant, preferably ascorbic acid or a salt thereof; citric acid (anhydrous and/or monohydrate) or a salt thereof; sodium metabisulfite, potassium metabisulfite or resveratrol; and
e. optionally, but according to a preferred embodiment, at least one water-soluble polymer with a molar mass of 5,000 to 500,000 g/mol, preferably 10,000 to 300,000 g/mol, even more preferred 15,000 to 200,000 g/mol even more preferred 20,000 to 200,000 g/mol, preferably hydroxypropyl methylcellulose, hydroxypropyl cellulose, or methylcellulose, more preferably hydroxypropyl methylcellulose (hypromellose).

Therefore, the present invention preferably relates to a liquid aqueous pharmaceutical composition as described above, comprising:
a. at least one pharmaceutically active compound in the form of a substituted glucopyranosyl-substituted benzene derivative or a pharmaceutically acceptable salt thereof, preferably 1-cyano-2-(4-cyclopropyl-benzyl)-4-(β-D-glucopyranos-1-yl)-benzene, or pharmaceutical acceptable salts thereof, more preferably 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline;
b. one or more etherified cyclodextrin derivatives in the form of etherified β-cyclodextrin, preferably hydroxyethyl-β-cyclodextrin, hydroxypropyl-β-cyclodextrin, dihydroxypropyl-β-cyclodextrin, more preferably hydroxypropyl-β-cyclodextrin (HPBCD);
c. one or more water-soluble preservatives, preferably selected from the group consisting of sorbic acid or salts thereof, benzoic acid or salts thereof, benzalkonium chloride, benzethonium chloride, cetylpyridinium chloride, sodium metabisulfite, sodium acetate; parabenes and salts thereof, preferably methylparabene, ethylparabene, propylparabene, butylparabene, butylparabene sodium; or combinations thereof, more preferably selected from the group consisting of sorbic acid or salts thereof, preferably sodium sorbate, potassium sorbate, calcium sorbate; benzoic acid or salts thereof, preferably sodium benzoate; benzalkonium chloride; benzethonium chloride; cetylpyridinium chloride; sodium metabisulfite; sodium acetate; or combinations thereof, most preferably sorbic acid or salts thereof;
d. optionally, but according to a preferred embodiment, at least one water-soluble antioxidant, preferably ascorbic acid or a salt thereof; citric acid (anhydrous and/or monohydrate) or a salt thereof; sodium metabisulfite, potassium metabisulfite or resveratrol; and
e. optionally, but according to a preferred embodiment, at least one water-soluble polymer with a molar mass of 5,000 to 500,000 g/mol, preferably 10,000 to 300,000 g/mol, even more preferred 15,000 to 200,000 g/mol, even more preferred 20,000 to 200,000 g/mol, preferably hydroxypropyl methylcellulose, hydroxypropyl cellulose, or methylcellulose, more preferably hydroxypropyl methylcellulose (hypromellose).

The liquid aqueous pharmaceutical composition according to the present invention preferably comprises:
a. 0.01 g/100 mL to 1 g/100 mL substituted benzimidazole or a pharmaceutically acceptable salt thereof, preferably pimobendan or a pharmaceutically acceptable salt thereof, or a substituted oxicam or a pharmaceutically acceptable salt thereof, preferably meloxicam or a pharmaceutically acceptable salt thereof, or a substituted imidazolinone or a pharmaceutically acceptable salt thereof, preferably 1-(4-chlorophenyl)-4-(4-morpholinyl)-2,5-dihydro-1H-imidazol-2-one (imepitoin) or a pharmaceutically acceptable salt thereof, or a substituted glucopyranosyl-substituted benzene derivative or a pharmaceutically acceptable form and/or salt thereof, preferably 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline;
b. 5 g/100 mL to 40 g/100 mL of one or more etherified cyclodextrin-derivatives, preferably hydroxypropyl-β-cyclodextrin;
c. 0.05 g/100 mL to 3.0 g/100 mL of at least one water-soluble preservative, preferably sorbic acid or a salt thereof;
d. optionally, but according to a preferred embodiment, 0.2 g/100 mL to 2.0 g/100 mL of at least one water-soluble antioxidant, preferably ascorbic acid or a salt thereof and
e. optionally, but according to a preferred embodiment, 0.01 g/100 mL to 0.75 g/100 mL of at least one water-soluble polymer, preferably hydroxypropyl methylcellulose (hypromellose).

According to another aspect the liquid aqueous pharmaceutical composition according to the present invention preferably comprises:
a. 0.1 g/100 mL to 0.25 g/100 mL pimobendan or a pharmaceutically acceptable salt thereof or meloxicam or a pharmaceutically acceptable salt thereof or 1-(4-chlorophenyl)-4-(4-morpholinyl)-2,5-dihydro-1H-imidazol-2-one (imepitoin) or a pharmaceutically acceptable salt thereof or 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline;
b. 20 g/100 mL to 35 g/100 mL of a hydroxypropyl-β-cyclodextrin;
c. 0.05 g/100 mL to 0.30 g/100 mL of hydroxypropyl methylcellulose (hypromellose);
d. 0.20 g/100 mL to 0.40 g/100 mL of a water-soluble preservative, preferably sorbic acid or a salt thereof;
e. 0.3 g/100 mL to 1.0 g/100 mL of an antioxidant, preferably ascorbic acid or a salt thereof;
f, wherein optionally the pH of the composition is between 2 to 10, preferably 3 to 10, more preferably 3 to 8, more preferably 3 to 7, more preferably 2.5 to 5, even more preferably 3 to 5, even more preferably 3.4 to 5 and most preferably 3.4 to 4.

With regard to the palatability if administered to animal patients the liquid aqueous pharmaceutically composition is well accepted.

The liquid aqueous pharmaceutical composition provides an acceptable solubility of the pharmaceutically active compound as disclosed herein, such as pimobendan in aqueous solution, according to which a minimum concentration of the pharmaceutically active compound is present allowing for use in an oral administration form. For example, the minimum concentration of pimobendan is preferably 1.5 mg/mL=0.15% (m/V). Furthermore, there is only a negligible crystal growth of the pharmaceutically active compound, if any, during the storage period. Further, the one or more water-soluble preservatives present assure the acceptable efficacy of microbial preservation. In addition, the chemical long-term stability of the active ingredient has been found to be good according to an accelerated stability test in the range of 3.0≤pH≤6.0.

The person skilled in the art knows the effective dosage of pharmaceutically active compounds as disclosed herein, such as benzimidazole derivatives, in particular pimobendan, and is readily able to adjust this dosage which is to be administered to the patient such as an animal patient, in need thereof. In order to have a general guidance in this connection a general therapeutic effective target dose, in particular for the treatment of HCM in cats, is about 0.1 mg to 0.5 mg pimobendan twice daily per kg bodyweight of the animal, preferably about 0.3 mg pimobendan twice daily per kg bodyweight of the animal.

The liquid aqueous pharmaceutical composition according to the present invention is intended for oral and/or parenteral administration, particularly oral solutions may be provided.

According to a preferred embodiment of the present invention the liquid aqueous pharmaceutical composition comprises the pharmaceutically active compound in form of a substituted benzimidazole, preferably pimobendan, or a substituted oxicam, preferably meloxicam, or a substituted imidazolinone, preferably 1-(4-chlorophenyl)-4-(4-morpholinyl)-2,5-dihydro-1H-imidazol-2-one (imepitoin) or a substituted glucopyranosyl-substituted benzene derivative, preferably 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline, in a therapeutically effective amount of up to 5 mg/mL, preferably of 1.5 to 4 mg/mL, even more preferably of 1.5 to 3 mg/mL.

According to a further aspect, the present invention also relates to a method of treatment and/or prevention of diseases, wherein cardiotonic, hypotensive, anti-inflammatory and anti-thrombotic substances have a therapeutic benefit, preferably directed to a subject suffering from heart diseases, particularly a hypertrophic cardiomyopathy, comprising the step of administering to such subject in need of such treatment a therapeutically effective amount of any of the liquid aqueous pharmaceutical compositions as described herein.

Preferably, the liquid aqueous pharmaceutical composition of the present invention is administered in a therapeutically effective amount from about 0.075 mg to about 0.5 mg in form of a substituted benzimidazole derivative, preferably pimobendan, or a substituted oxicam, preferably meloxicam, or a substituted imidazolinone preferably 1-(4-chlorophenyl)-4-(4-morpholinyl)-2,5-dihydro-1H-imidazol-2-one (imepitoin) or a substituted glucopyranosyl-substituted benzene derivative, preferably 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline, per kg bodyweight of the animal, more preferably from about 0.2 mg to about 0.4 mg of the pharmaceutically active compound in form of a substituted benzimidazole derivative, preferably pimobendan, or a substituted oxicam, preferably meloxicam, or a substituted imidazolinone preferably 1-(4-chlorophenyl)-4-(4-morpholinyl)-2,5-dihydro-1H-imidazol-2-one (imepitoin) or a substituted glucopyranosyl-substituted benzene derivative, preferably 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline, per kg bodyweight of the animal, even more preferably about 0.3 mg of the pharmaceutically active compound in form of a substituted benzimidazole derivative, preferably pimobendan, or a substituted oxicam, preferably meloxicam, or a substituted imidazolinone preferably 1-(4-chlorophenyl)-4-(4-morpholinyl)-2,5-dihydro-1H-imidazol-2-one (imepitoin) or a substituted glucopyranosyl-substituted benzene derivative, preferably 1-cyano-2-(4-cyclopropyl-benzyl)-4-(β-D-glucopyranos-1-yl)-benzene, or any pharmaceutically acceptable form and/or salt thereof, wherein the pharmaceutically acceptable form preferably is a crystalline complex between 1-cyano-2-(4-cyclopropyl-benzyl)-4-(β-D-glucopyranos-1-yl)-benzene and one or more amino acids, preferably wherein the one or more amino acids is proline, more preferably L-proline, twice daily per kg bodyweight of the animal. Preferably, two doses are to be administered per day (twice daily administration).

The subject/patient in need of any such treatment mentioned above is a mammal, preferably a companion animal. The term “animal” as used herein includes but is not limited to companion animals such as dogs, cats, guinea pigs, hamsters, horses, cattle, goats, sheep or the like. Preferably, the subject in need of such treatment is a dog, horse or cat, most preferably a cat or dog.

The liquid aqueous pharmaceutical composition according to the present invention is for use in a method for treating a patient in need of such treatment, preferably selected from among the indications: heart failure (HF), congestive heart failure (CHF), acute CHF, decompensated endocardiosis (DCE), dilated cardiomyopathy (DCM), asymptomatic (occult) CHF, asymptomatic DCM, hypertrophic cardiomyopathy (HCM), restricted cardiomyopathy (RCM), and heart failure due to HCM, RCM, DCM and/or UCM.

More preferably, the liquid aqueous pharmaceutical composition according to the present invention is for use in a method for treating a subject in need of such treatment, preferably an animal, in particular a companion animal, even more preferred horse, dog or cat, guinea pig, hamster, cattle, goat, sheep, in particular cat or dog, selected from among the indications: heart diseases, particularly a hypertrophic cardiomyopathy, more particularly heart failure (HF), congestive heart failure (CHF), acute CHF, decompensated endocardiosis (DCE), dilated cardiomyopathy (DCM), asymptomatic (occult) CHF, asymptomatic DCM, hypertrophic cardiomyopathy (HCM), restricted cardiomyopathy (RCM), and heart failure due to HCM, RCM, DCM and/or UCM.

The present invention is also directed to the use of a liquid aqueous pharmaceutical composition as above defined for preparing a pharmaceutical composition for the treatment or prevention of diseases in a subject in need of such treatment, preferably selected from among the above indications.

In a preferred embodiment, the liquid aqueous pharmaceutical composition as defined above for use in the above mentioned methods is for oral and/or parenteral administration, preferably oral administration.

Also subject of the present invention is a kit of parts that comprises:
a. a preserved liquid aqueous pharmaceutical composition as described above; and
b. a package leaflet including the information that the pharmaceutical composition is to be used for the prevention and/or treatment of a heart disease, preferably heart failure and/or hypertrophic cardiomyopathy, in a subject in need of such prevention or treatment.

During the production it has been surprisingly found that it is preferable that the one or more water-soluble preservatives are added after the addition of the at least one pharmaceutically active compound as disclosed herein. In case the one or more water-soluble preservatives are added to the cyclodextrin mixture before the at least one pharmaceutically active compound, the solution may become turbid. If the one or more water-soluble preservatives are added after the at least one pharmaceutically active compound, the produced solution remains clear.

According to a further aspect, the present invention also relates to a manufacturing process for the production of any of the liquid aqueous pharmaceutical compositions as described herein. A process for producing the pharmaceutical composition comprises the steps of: adding at least one pharmaceutically active compound, one or more etherified cyclodextrin derivatives, one or more water-soluble preservatives, optionally one or more antioxidants and optionally at least one water-soluble polymer to water and mixing under stirring, adjusting the pH value using a pH adjustment agent, wherein preferably the one or more water-soluble preservatives are added after the addition of the at least one pharmaceutically active compound.

In this regard it should be taken into account that the process of manufacturing may be arbitrarily selected from manufacturing processes of liquid pharmaceutical compositions known from prior art unless the one or more water-soluble preservatives are added after the addition of the at least one pharmaceutically active compound.

In the following a representative process is described which should not be construed to limit the present invention.

At first, water is weighed in. Optionally, the at least one water-soluble polymer is added, preferably in portions, to the water under stirring until the at least one water-soluble polymer is dissolved thereby obtaining a first liquid mixture. Alternatively, the one or more etherified cyclodextrin derivatives are added to the water under stirring thereby obtaining a first liquid mixture. Alternatively and optionally, the one or more etherified cyclodextrin derivatives are added to the first liquid mixture containing the at least one water-soluble polymer under stirring until the one or more etherified cyclodextrin derivatives are dissolved thereby obtaining a first liquid mixture. Then, an ultrasonic treatment of such first liquid mixture or, preferably under stirring, may be optionally performed. The obtained first liquid mixture or is incubated at room temperature, preferably without stirring, for one or more minutes. Afterwards, the at least one pharmaceutically active compound is added, preferably in portions, under stirring until it is dissolved thereby obtaining a second liquid mixture. Subsequently, the one or more water-soluble preservatives are added, preferably in portions, to the obtained second liquid mixture under stirring until they are dissolved thereby obtaining a third liquid mixture. Optionally, one or more antioxidants as well as further excipients, if so desired, are added, preferably in portions, to the third liquid mixture during stirring thereby obtaining a fourth liquid mixture. Then, an ultrasonic treatment of the fourth liquid mixture, preferably under stirring, is optionally performed. The obtained fourth liquid mixture is incubated at room temperature, preferably without stirring, for one or more minutes. Subsequently, the pH value of the obtained fourth liquid mixture is determined and adjusted, if necessary, using a pH adjustment agent to the desired pH value thereby obtaining the liquid aqueous pharmaceutical composition of the present invention.

The at least one pharmaceutically active compound, one or more etherified cyclodextrin derivatives, one or more water-soluble preservatives, and one or more antioxidants and at least one water-soluble polymer are those as already described in detail supra. The pH adjustment agent is preferably hydrochloric acid and/or sodium hydroxide.

The amounts used depend from the at least one pharmaceutically active compound used as well as the intended treatment, administration route and the patient to be treated. The person skilled in the art is readily able to select and adjust the required amounts by his general knowledge.

The invention described will now be illustrated by figures. However, it is expressly pointed out that the figures are intended solely as an illustration and should not be regarded as restricting the invention.

### BRIEF DESCRIPTION OF THE FIGURES

Further advantages, features, characteristics and aspects of the present invention arise from the drawings which show as follows:

FIG. 1 a schematic diagram wherein the solubility of pimobendan is shown as a function of preservative, cyclodextrin type and pH value in solutions containing 25% (m/V) cyclodextrin;

FIG. 2 a schematic diagram wherein the solubility of pimobendan is shown as a function of type and concentration of polymer, salt or complexation agent; and

FIG. 3 a schematic diagram wherein the solubility of pimobendan is shown as a function of concentration of hydroxypropyl-β-cyclodextrin and presence of sodium sorbate and hydroxypropyl methylcellulose (HPMC).

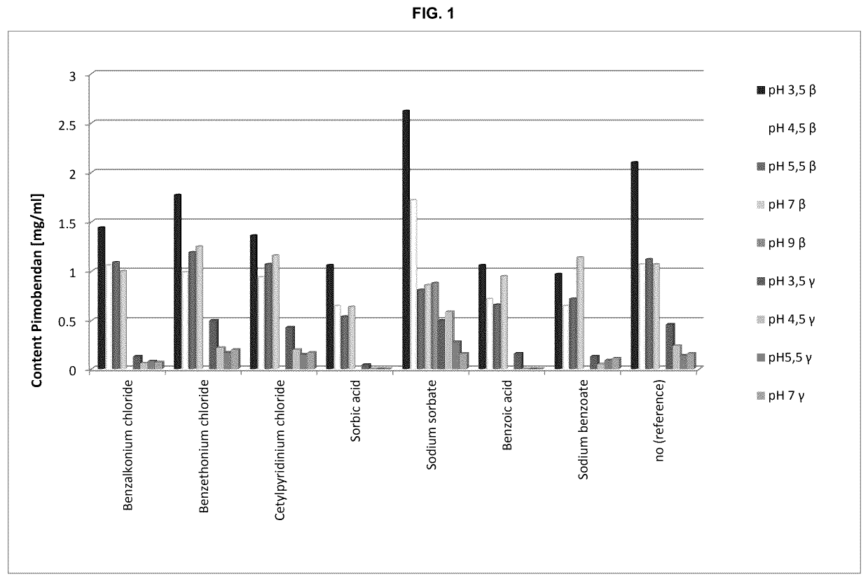

FIG. 1 shows a schematic diagram wherein the solubility of pimobendan is indicated as a function of the water-soluble preservatives benzalkonium chloride, benzethonium chloride, cetalpyridinium chloride, sorbic acid, sodium sorbate, benzoic acid, and sodium benzoate, respectively. The last row of columns represents the reference control, which is the respective solution without preservative (“none”).

Each water-soluble preservative has been used with pH values of 3.5, 4.5, 5.5, 7, and 9 in combination with a hydroxypropyl-β-cyclodextrin abbreviated as “β” and each water-soluble preservative has been used with pH values of 3.5, 4.5, 5.5, and 7 in combination with a hydroxypropyl-gamma-cyclodextrin abbreviated as “γ”. The solutions contain 25% (m/V) cyclodextrin. Each column in the diagram shows the determined solubility of pimobendan as a function of preservative, cyclodextrin type and pH value.

In FIG. 1 it can be seen that the highest solubility of pimobendan occurs at pH=3.5. Furthermore, pimobendan is more soluble with hydroxypropyl-β-cyclodextrin than hydroxypropyl-gamma-cyclodextrin. The highest pimobendan solubility is achieved with sodium sorbate for which the solubility is significantly higher compared with the results of the reference control wherein no preservative is present.

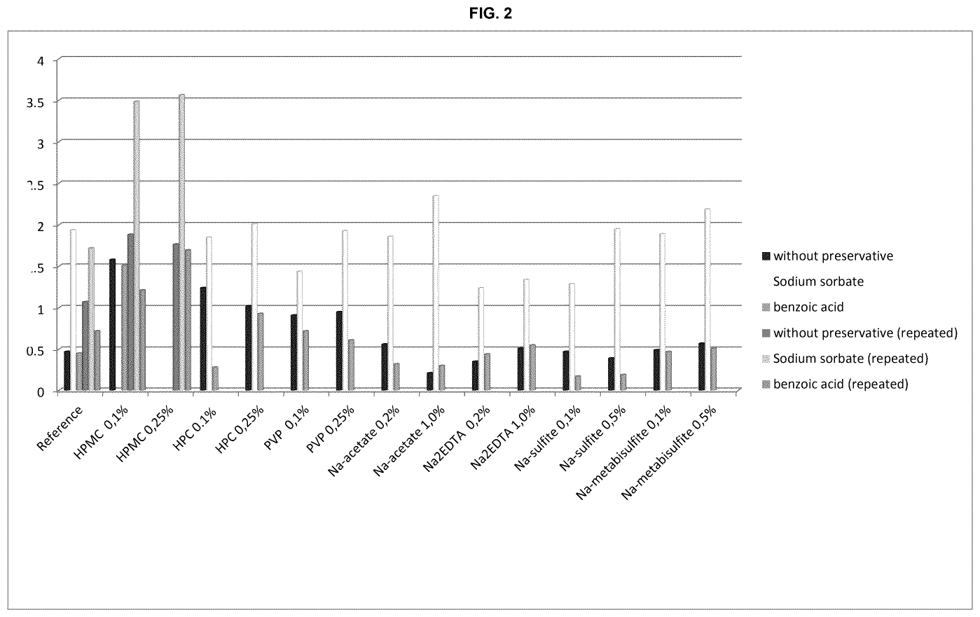

FIG. 2 is a schematic diagram wherein the solubility of pimobendan is shown as a function of type and concentration of polymer, salt or complexation agent. In order to determine the degree of complexation, the effect of three different polymers, three different salts and one chelating agent on the solubility of pimobendan was tested.

The pH of the solution was 4.5. Metolose is hydroxypropyl methylcellulose=HPMC=Hypromellose. Klucel ELF is hydroxypropyl cellulose=HPC. The number after the chemical name indicates the concentration of additive in % (m/V).

The consistency of the reference values [e.g. “sodium sorbate” vs. “sodium sorbate (repeated)”] shows that the results are consistent between the different trials and serves as a plausibility check.

In FIG. 2 it can be seen that the addition of HPMC results in a significant increase in the solubility of pimobendan. The addition of salts or disodium edetate does not significantly increase the solubility of pimobendan.

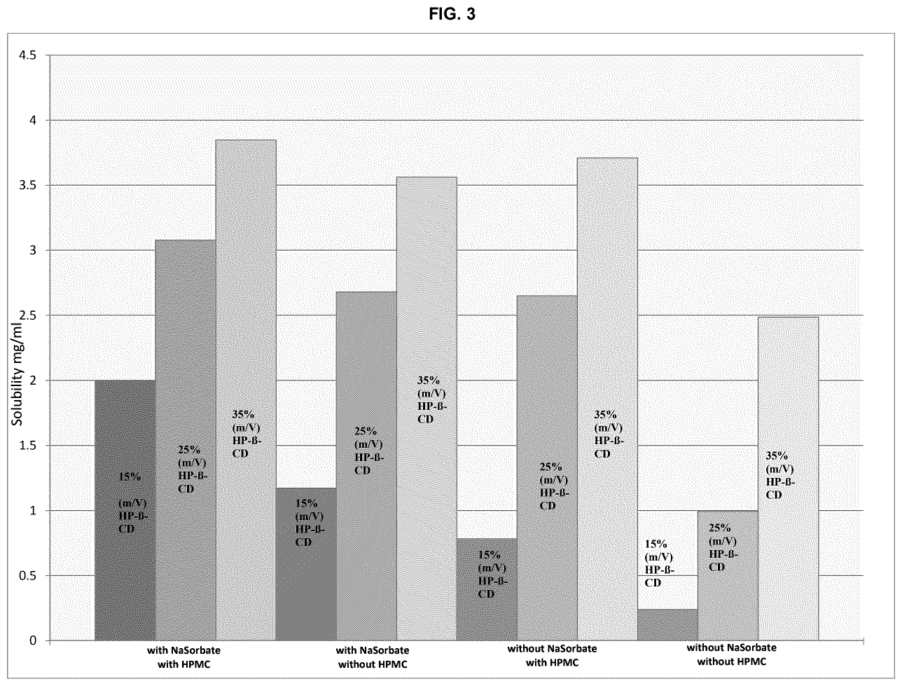

FIG. 3 is a schematic diagram wherein the solubility of pimobendan is shown as a function of concentration of hydroxypropyl-β-cyclodextrin and presence of sodium sorbate and hydroxypropyl methylcellulose (HPMC). Therefore, in FIG. 3 the effect of sodium sorbate and HPMC on the solubility of pimobendan was illustrated, and also the effect of concentration of hydroxypropyl-β-cyclodextrin on the pimobendan solubility. Concentrations of sodium sorbate of 1.0% (m/V) and of HPMC of 0.1% (m/V) were used. The pH value was set to 4.5 using hydrochloric acid in all solutions.

In FIG. 3 it can be seen that the results confirm that sodium sorbate significantly increases the solubility of pimobendan. Furthermore, the results also confirm that HPMC significantly increases the solubility of pimobendan. By use of both HPMC and sodium sorbate the solubility of pimobendan is significantly increased.

The invention described will now be illustrated by Examples. However, it is expressly pointed out that the Examples and description are intended solely as an illustration and should not be regarded as restricting the invention. In the following the invention shall be illustrated in form of exemplary pharmaceutical compositions. However, the present invention is not limited to the described compositions, but other components, amounts and additives are possible.

### EXAMPLES

### Example 1: Manufacturing Process

In the following Table 1 exemplary pharmaceutical compositions according to the present invention are given in detail:

TABLE 1
Exemplary pharmaceutical compositions according to the present
invention

| Ingredient | Content [g/100 mL] | Function |
|---|---|---|
| Pimobendan | 0.15-0.25 | Pharmaceutically |
| | | active compound |
| Hydroxypropyl-β- | 15-35 | Etherified cyclodextrin |
| cyclodextrin | | |
| Hydroxypropyl | 0.05-2.5 | Water-soluble polymer |
| methylcellulose | | |
| Sorbic acid and/or | 0.1-1.0 | Water-soluble |
| potassium sorbate | | preservative |
| sodium benzoate | | |
| sodium metabisulfite | | |
| Ascorbic acid and/or | 0.05-1.0 | Antioxidant |
| sodium ascorbate | | |
| sodium metabisulfite | | |
| citric acid | | |
| sodium citrate | | |
| Hydrochloric acid 0.1M | ad pH 3.1-4.0 | pH adjustment |
| Water | ad 100 mL | Solvent |

The production procedure of an exemplary pharmaceutical composition according to the present invention for a single small scale batch (100 mL) with a target pH value of 3.5 in form of a general instruction is as follows:
1. Weigh purified water. Add a magnetic stirrer.
2. Weigh hydroxypropyl methylcellulose (HPMC) and add in portions to the purified water while stirring.
3. Weigh hydroxypropyl-β-cyclodextrin into a 100 mL glass bottle and add the HPMC solution while stirring until the hydroxypropyl-β-cyclodextrin is dissolved.
4. Let incubate at room temperature without stirring for 10 minutes.
5. Weigh pimobendan and add in portions while stirring until pimobendan is dissolved.
6. Weigh sorbic acid and add in portions while stirring until sorbic acid is dissolved.
7. Weigh ascorbic acid and optionally free-radical scavengers (e.g. BHA or propyl gallate) and add in portions while stirring and nitrogen atmosphere until ascorbic acid and optionally free-radical scavengers are dissolved.

8. Let incubate at room temperature without stirring for 10 minutes.

9. Determine pH and, if necessary, adjust to 3.50.

### Example 2: Antimicrobial Efficacy

The testing criteria applied are those for evaluation of antimicrobial activity for oral preparations according to Pharm. Eur. 7 (tests at 14 days and 28 days). The acceptance criteria of the Ph. Eur. 7, Method 5.1.3 “Efficacy of Antimicrobial Preservation” USP 34, and Method <51> Antimicrobial Effectiveness Testing are listed in the following Table 2.

TABLE 2
Criteria for evaluation of antimicrobial activity for oral preparations
according to Pharm. Eur. 7 and USP 34

| | Ph. Eur. 7 Method 5.1.3. | | USP 34 Method <51> | |
|---|---|---|---|---|
| Type of micro- | Logarithmic reduction of microorganisms after | | | |
| organism | 14 days | 28 days | 14 days | 28 days |
| Bacteria | >3 | No increase | >1.0 | No increase |
| | | from 14 days ¹⁾ | | from 14 days ²⁾ |
| Fungi | >1 | No increase | No increase | No increase |
| | | from 14 days ¹⁾ | from initial | from initial |
| | | | calc. count ²⁾ | calc. count ²⁾ |

¹⁾ for Ph. Eur: No increase = no increase in number
²⁾ for USP: No increase = not more than 0.5 log₁₀ units higher than reference value

The formulations tested in the trial are shown in the following Table 3.

The following microorganisms were tested: Pseudomonas aeruginosa, Straphylococcus aureus, Escherichia coli, Candida albicans, Aspergillus brasiliensis, Zygosaccharomyces rouxi.

TABLE 3

| | Formulation no. | | | | | | | | | | | | | | | | | | | |
|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 |
| Components | Concentration [g/100 mL] | | | | | | | | | | | | | | | | | | | |
| Pimobendan | 0.15 | | | | | | | | | | | | | | | | | | | |
| HP-β-CD | 25 | | | | | | | | | | | | | | | | | | | |
| HPMC | 0.1 | | | | | | | | | | | | | | | | | | | |
| Sorbic acid | 0.1 | 0.2 | 0.3 | 0.4 | 0.5 | 0.6 | 0.7 | 0.8 | 0.2 | 0.2 | 0.4 | 0.4 | — | — | — | — | — | — | — | — |
| Calcium | — | — | — | — | — | — | — | — | 0.2 | — | 0.4 | — | — | — | — | — | — | — | — | — |
| sorbate | | | | | | | | | | | | | | | | | | | | |
| Potassium | — | — | — | — | — | — | — | — | — | 0.2 | — | 0.4 | — | — | — | — | — | — | — | — |
| sorbate | | | | | | | | | | | | | | | | | | | | |
| Sodium | — | — | — | — | — | — | — | — | — | — | — | — | 0.2 | 0.4 | 0.6 | 0.8 | 1.0 | 0.8 | — | — |
| benzoate | | | | | | | | | | | | | | | | | | | | |
| Benzalk. | — | — | — | — | — | — | — | — | — | — | — | — | — | — | — | — | — | — | 0.1 | 0.1 |
| chloride | | | | | | | | | | | | | | | | | | | | |
| Ascorbic | 0.05 | 0.05 | 0.05 | 0.05 | 0.05 | 0.05 | 0.05 | 0.05 | 0.05 | 0.05 | 0.05 | 0.05 | — | — | — | — | — | 0.05 | — | 0.05 |
| acid | | | | | | | | | | | | | | | | | | | | |

| | |
|---|---|
| HCl q.s. | ad pH 3.5 |
| Purified | ad 100 mL |
| water | |

In the performed tests the USP 34 Method <51> Criteria as listed in Table 2 were found to be fulfilled for all solutions for all microorganisms.

### Example 3: Formulation Samples were Produced with Compositions Listed in the Following Table 4

TABLE 4

| | Formulation No. | | | | | | | | |
|---|---|---|---|---|---|---|---|---|---|
| | 1 | 2 | 3 | 4 | 5 | 6 | 7 | 8 | 9 |
| Ingredient | Concentration [g/100 mL] | | | | | | | | |
| Pimobendan | — | — | _ | — | — | — | — | 0.15 | 0.15 |
| 1-cyano-2-(4- | 0.1 | 0.1 | 0.1 | — | — | — | — | — | — |
| cyclopropyl- | | | | | | | | | |
| benzyl)-4-(β-D- | | | | | | | | | |
| glucopyranos-1-yl)- | | | | | | | | | |
| benzene L-Proline | | | | | | | | | |
| Imepitoin | — | — | — | 0.1 | 0.1 | 0.1 | | | |
| Meloxicam | — | — | — | — | — | — | 0.1 | — | — |
| Hydroxypropyl-ß- | 25 | 25 | 25 | 25 | 25 | 25 | 25 | 25 | 25 |
| cyclodextrin | | | | | | | | | |
| Hydroxypropyl | — | 0.1 | 0.1 | — | 0.1 | 0.1 | 0.1 | 0.1 | 0.1 |
| methylcellulose | | | | | | | | | |
| Sorbic acid | 0.3 | — | 0.3 | 0.3 | — | 0.3 | 0.3 | — | — |
| Methyl paraben | — | — | — | — | — | — | — | 0.18 | 0.18 |
| Propyl paraben | — | — | — | — | — | — | — | 0.02 | 0.02 |
| HCl q.s. ad | pH 3.5 | pH 3.5 | pH 3.5 | pH 3.5 | pH 3.5 | pH 3.5 | pH 3.5 | pH 3.5 | pH 5.0 |

| | |
|---|---|
| Water, purified | add 100 mL |

The following procedure was used to prepare the samples:
1. Weigh entire amount of water into vessel.
2. Weigh entire amount of hydroxypropyl methylcellulose (HPMC) into a beaker and add slowly to stirred water. Stir until fully dissolved.
3. Weigh entire amount of Hydroxypropyl-β-cyclodextrin (HPBCD) into a beaker and add slowly to stirred mixture. Stir until fully dissolved.
4. Let solution stand at least 10 minutes.
5. Weigh entire amount of drug substance into a beaker and add slowly to stirred mixture. Stir until fully dissolved.
6. Weigh entire amount of sorbic acid into a beaker and add slowly to stirred mixture. Stir until fully dissolved.
7. Let solution stand at least 10 minutes.
8. Adjust pH to target value with HCl or NaOH.
9. Let solutions stand overnight and re-adjust pH to target value with HCl or NaOH.

The solutions were found to have the following densities and appearances:

TABLE 5

| Formulation/Solution | Density [g/mL] | Appearance |
|---|---|---|
| 1 | 1.082 | Clear, colorless, no particles |
| 2 | 1.096 | Clear, colorless, no particles |
| 3 | 1.076 | Clear, colorless, no particles |
| 4 | 1.075 | Clear, colorless, no particles |
| 5 | 1.094 | Clear, colorless, no particles |
| 6 | 1.085 | Clear, colorless, no particles |
| 7 | 1.074 | Clear, light yellow, no particles |
| 8 | 1.080 | Clear, colorless, no particles |
| 9 | 1.082 | Clear, colorless, no particles |

TABLE 6
Microbial results for tested solutions: E. coli (bacterium), P. aeruginosa
(bacterium) and S. aureus (bacterium).

| Micro- | Colony forming units/g | | | | |
|---|---|---|---|---|---|
| organism | Solution no. | Innoc. | 7 days | 14 days | 28 days |
| Escherichia coli | 1 | 540 000 | <100 | <100 | no data available |
| | 2 | 540 000 | 7300 | 500 | no data available |
| | 3 | 540 000 | <100 | <100 | no data available |
| | 4 | 540 000 | <100 | <100 | no data available |
| | 5 | 540 000 | 48 000 | 2900 | no data available |
| | 6 | 540 000 | <100 | <100 | no data available |
| | 7 | 420 000 | <100 | no data available | no data available |
| | 8 | Test not possible ¹⁾ | | | |
| | 9 | Test not possible ¹⁾ | | | |
| Pseudomonas aeruginosa | 1 | 440 000 | <100 | <100 | no data available |
| | 2 | 440 000 | <100 | <100 | no data available |
| | 3 | 440 000 | <100 | <100 | no data available |
| | 4 | 440 000 | <100 | <100 | no data available |
| | 5 | 440 000 | ²⁾ | 16 000 | no data available |
| | 6 | 440 000 | <100 | <100 | no data available |
| | 7 | 500 000 | <100 | no data available | no data available |
| | 8 | Test not possible ¹⁾ | | | |
| | 9 | Test not possible ¹⁾ | | | |
| Staphylococcus aureus | 1 | 350 000 | <100 | <100 | no data available |
| | 2 | 350 000 | <100 | <100 | no data available |
| | 3 | 350 000 | <100 | <100 | no data available |
| | 4 | 350 000 | <100 | <100 | no data available |
| | 5 | 350 000 | <100 | <100 | no data available |
| | 6 | 350 000 | <100 | <100 | no data available |
| | 7 | 320 000 | <100 | no data available | no data available |

| | |
|---|---|
| 8 | Test not possible ¹⁾ |
| 9 | Test not possible ¹⁾ |

¹⁾ Test could not be started due to rapid microbial growth between filtration and start of test
²⁾ Result not reliable due to high count

TABLE 7
Microbial results for tested solutions: Z. rouxii (yeast fungus),
C. albicans (yeast fungus) and A. brasiliensis (mold fungus).

| Micro- | Colony forming units/g | | | | |
|---|---|---|---|---|---|
| organism | Solution no. | Innoc. | 7 days | 14 days | 28 days |
| Candida albicans | 1 | 380 000 | <100 | <100 | no data available |
| | 2 | 380 000 | 530 000 | 790 000 | no data available |
| | 3 | 380 000 | <100 | <100 | no data available |
| | 4 | 380 000 | <100 | <100 | no data available |
| | 5 | 380 000 | 500 000 | 660 000 | no data available |
| | 6 | 380 000 | <100 | <100 | no data available |
| | 7 | 370 000 | <100 | no data available | no data available |
| | 8 | Test not possible ¹⁾ | | | |
| | 9 | Test not possible ¹⁾ | | | |
| Aspergillus brasiliensis | 1 | 120 000 | <100 | 100 | no data available |
| | 2 | 120 000 | 290 000 | 190 000 | no data available |
| | 3 | 120 000 | 7800 | <100 | no data available |
| | 4 | 120 000 | 10 000 | 600 | no data available |
| | 5 | 120 000 | 830 0000 | 740 000 | no data available |
| | 6 | 120 000 | 3000 | 700 | no data available |
| | 7 | 290 000 | 600 | no data available | no data available |

| | |
|---|---|
| 8 | Test not possible ¹⁾ |
| 9 | Test not possible ¹⁾ |

¹⁾ Test could not be started due to rapid microbial growth between filtration and start of test

It is seen from the results in Table 6 and 7 that a good antimicrobial efficacy is achieved through the use of sorbic acid as a water-soluble preservative. The solutions (no. 2 and 5) with no water-soluble preservative fail the criteria for evaluation of antimicrobial activity according to Ph. Eur. The solutions with methyl paraben and propyl paraben (no. 8 and 9) had such a high microbial growth that the test of antimicrobial efficacy was not possible.

### Example 4: Small Amounts of Antioxidant, for Example Ascorbic Acid, Surprisingly Provided an Improvement of the Efficacy of Microbial Preservation

TABLE 8
Formulation compositions in test of efficacy of microbial preservation.

| | Formulation no. | | | |
|---|---|---|---|---|
| | 1 | 2 | 3 | 4 |
| Ingredient | Concentration [g/100 mL] | | | |
| Pimobendan | 0.15 | 0.15 | 0.15 | 0.15 |
| Hydroxypropyl-ß- | 25 | 25 | 25 | 25 |
| cyclodextrin | | | | |
| Hydroxypropyl | 0.1 | 0.1 | 0.1 | 0.1 |
| methylcellulose | | | | |
| Sorbic acid | 0.3 | 0.3 | 0.3 | 0.3 |
| Ascorbic acid | 0.20 | 0.35 | 0.50 | 0.70 |
| HCl q.s. ad | pH 3.5 | pH 3.5 | pH 3.5 | pH 3.5 |

| | |
|---|---|
| Water, purified | ad 100 mL |

TABLE 9
Microbiological results according to Pharm. Eur. Method 2.6.12. for the
fungi Zygosaccharomyces rouxii, Candida albicans and Aspergillus
brasiliensis with varying concentrations of ascorbic acid.

| | Formulation no./incubation period (days) | | | | | | | |
|---|---|---|---|---|---|---|---|---|
| | 1 | | 2 | | 3 | | 4 | |
| | | 28 | | 28 | | 28 | | 28 |
| Micro-organism | 14 d | d | 14 d | d | 14 d | d | 14 d | d |
| Zygosaccharomyces | a | a | a | a | a | a | a | a |
| rouxii | | | | | | | | |
| Candida albicans | b | a | a | a | a | a | a | a |
| Aspergillus | c | c | c | b | b | b | b | a |
| brasiliensis | | | | | | | | |

Codes:
a: <LOQ CFU/mL,
b: LOQ-1000 CFU/mL,
c: >1000-10 000 CFU/mL,
where CFU = colony forming units and LOQ = limit of quantification

The above results demonstrate the increasing efficacy of preservation with increasing concentration of antioxidant, such as ascorbic acid.

### Example 5: The Formulations According to EP 1 920 785, Paragraph were Produced (See Table 10)

TABLE 10

| | mg/10 ml | |
|---|---|---|
| Material | Formulation #1 | Formulation #2 |
| Pimobendan | 10.0 | 7.5 |
| Kleptose HP (HP | 3300.0 | 3000.0 |
| Disodium hydrogen | 17.6 | 17.6 |
| phosphate dodecahydrate | | |
| Sodium dihydogen | 8.0 | 8.0 |
| phosphate dihydrate | | |
| Methyl paraben | 20.0 | 10.0 |
| Propyl paraben | 5.0 | 5.0 |
| Disodium edetate | 5.0 | 5.0 |
| Water for injection | q.s. to 10 ml | q.s. to 10 ml |

Both formulations were clear, colorless and showed no particles. Formulation #1 has a measured pH of 8.2. Formulation #2 has a measured pH of 7.6.

What is claimed:

1. A method for treating an animal in need of treatment of an indication, the indication selected from the group consisting of hypertrophic cardiomyopathy, heart failure (HF), congestive heart failure (CHF), acute CHF, decompensated endocardiosis (DCE), dilated cardiomyopathy (DCM), asymptomatic (occult) CHF, asymptomatic DCM, hypertrophic cardiomyopathy (HCM), restricted cardiomyopathy (RCM), and heart failure due to HCM, RCM, DCM and/or UCM, the animal being selected from the group consisting of a horse, a dog, a cat, a guinea pig, a hamster, cattle, a goat, and a sheep, the method comprising administering a preserved liquid aqueous pharmaceutical composition to the animal, wherein the preserved liquid aqueous pharmaceutical composition comprises:
at least one etherified cyclodextrin derivative in an amount of 5 g/100 ml to 40 g/100 ml;
at least one water-soluble preservative in an amount of 0.1 g/100 ml to 3.0 g/100 ml, wherein the at least one water-soluble preservative comprises sorbic acid or a salt thereof or sorbic acid or a salt thereof in combination with one or more selected from the group consisting of benzoic acid or a salt thereof, benzalkonium chloride, benzethonium chloride, cetylpyridinium chloride, sodium metabisulfite, sodium acetate, and a parabene or a salt thereof;
at least one water-soluble antioxidant in an amount of 0.05 g/100 ml to 1.0 g/100 ml, the at least one water-soluble antioxidant comprising ascorbic acid or a pharmaceutically acceptable salt thereof or ascorbic acid or a pharmaceutically acceptable salt thereof in combination with one or more selected from the group consisting of citric acid (anhydrous and/or monohydrate) or a pharmaceutically acceptable salt thereof, erythorbic acid, fumaric acid, malic acid, monothioglycerol, phosphoric acid, sodium metabisulfite, potassium metabisulfite, propionic acid, sodium bisulfite, sodium sulfite, resveratrol, butylhydroxyanisol, and a gallate derivative;
at least one water soluble-polymer in an amount of 0.01 g/100 ml to 0.75 g/100 ml; and
at least one pharmaceutically active compound in an amount of 0.01 g/100 ml to 1.0 g/100 ml, the at least one pharmaceutically active compound comprising a benzimidazole derivative.

2. The method of claim 1, wherein:
the benzimidazole derivative is selected from the group consisting of thiabendazol, fuberidazol, oxibendazol, parbendazol, cambendazol, mebendazol, fenbendazol, flubendazol, albendazol, oxfendazol, nocodazol, astemisol, pimobendan, and pharmaceutically acceptable salts, derivatives, metabolites or pro-drugs thereof.

3. The method of claim 1, wherein the at least one pharmaceutically active compound comprises pimobendan.

4. The method of claim 3, wherein the preserved liquid aqueous pharmaceutical composition is administered to the animal in a daily dosage amount of pimobendan that is 0.1 mg/kg bodyweight to 0.5 mg/kg bodyweight twice daily.

5. The method of claim 1, wherein the preserved liquid aqueous pharmaceutical composition is administered orally.

6. The method of claim 1, wherein the at least one water-soluble antioxidant comprises one or more selected from the group consisting of sodium ascorbate, sodium citrate, and propylgallate.

7. The method of claim 1, wherein the at least one water-soluble preservative is present in an amount no greater than 1.0 g/100 ml.

8. The method of claim 1, wherein the preserved liquid aqueous pharmaceutical composition further comprises at least one water-soluble polymer having a molar mass of 5,000 to 500,000 g/mol.

9. The method of claim 8, wherein the at least one water soluble-polymer is present in an amount from 0.01 g/100 ml to 0.75 g/100 ml.

10. The method of claim 8, wherein the at least one water-soluble polymer is selected from the group consisting of hydroxypropyl methylcellulose (hypromellose, HPMC), hydroxypropyl cellulose, carboxymethylcellulose, hydroxyethyl cellulose, hydroxyethylmethyl cellulose, ethylcellulose, methylcellulose, polyvinylpyrrolidone, polyvinylacetate and combinations or copolymers thereof.

11. The method of claim 1, wherein the at least one etherified cyclodextrin derivative comprises etherified β-cyclodextrin having the chemical formula I: 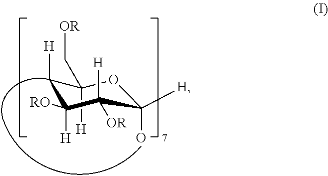
in which the residues R are independently from each other hydroxyalkyl groups.

12. The method of claim 1, wherein the at least one etherified cyclodextrin derivative comprises one or more selected from the group consisting of hydroxyethyl-β-cyclodextrin, hydroxypropyl-β-cyclodextrin, dihydroxypropyl-β-cyclodextrin, and sulphobutyl ether-β-cyclodextrin.

13. The method of claim 1, wherein the preserved liquid aqueous pharmaceutical composition further comprises sodium metabisulfite, potassium metabisulfite or resveratrol.

14. A method for treating an animal in need of treatment of an indication, the indication selected from the group consisting of hypertrophic cardiomyopathy, heart failure (HF), congestive heart failure (CHF), acute CHF, decompensated endocardiosis (DCE), dilated cardiomyopathy (DCM), asymptomatic (occult) CHF, asymptomatic DCM, hypertrophic cardiomyopathy (HCM), restricted cardiomyopathy (RCM), and heart failure due to HCM, RCM, DCM and/or UCM, the animal being selected from the group consisting of a horse, a dog, a cat, a guinea pig, a hamster, cattle, a goat, and a sheep, the method comprising administering a preserved liquid aqueous pharmaceutical composition to the animal, wherein the preserved liquid aqueous pharmaceutical composition comprises:
pimobendan, or pharmaceutically acceptable salts thereof;
at least one etherified cyclodextrin derivative comprising hydroxypropyl-β-cyclodextrin (HPβCD);
at least one water-soluble antimicrobial preservative comprising sorbic acid in an amount of 0.20 g/100 mL to 0.40 g/100 mL;
at least one water-soluble antioxidant comprising ascorbic acid in an amount of 0.3 g/100 mL to 1.0 g/100 mL; and
hydroxypropyl methylcellulose (hypromellose);
wherein a pH of the composition is from 2.5 to 5, and the composition is free of paraben and sodium edetate.

15. The method of claim 14, wherein the preserved liquid aqueous pharmaceutical composition is administered to the animal in a daily dosage amount of pimobendan that is 0.1 mg/kg bodyweight to 0.5 mg/kg bodyweight twice daily.

16. The method of claim 14, wherein the preserved liquid aqueous pharmaceutical composition is administered orally.

17. The method of claim 14, wherein the pH of the preserved liquid aqueous pharmaceutical composition is from 3 to 5.

18. The method of claim 14, wherein the pH of the preserved liquid aqueous pharmaceutical composition is from 3.4 to 4.

19. The method of claim 1, wherein the preserved liquid aqueous pharmaceutical composition further comprises:
0.1 g/100 mL to 0.25 g/100 mL pimobendan or pharmaceutically acceptable salts thereof;
20 g/100 mL to 35 g/100 mL of hydroxypropyl-β-cyclodextrin (HPBCD); and
0.05 g/100 mL to 0.30 g/100 mL of hydroxypropyl methylcellulose (hypromellose).

20. A process for producing a preserved liquid aqueous pharmaceutical composition, comprising:
adding pimobendan or pharmaceutical acceptable salts thereof, hydroxypropyl-β-cyclodextrin (HPBCD), sorbic acid or pharmaceutically acceptable salts thereof, ascorbic acid or pharmaceutically acceptable salts thereof and hydroxypropyl methylcellulose (hypromellose) to water and mixing under stirring; and
adjusting the pH value using a pH adjustment agent;
wherein the sorbic acid or pharmaceutically acceptable salts thereof are added after the addition of pimobendan or pharmaceutical acceptable salts thereof.
